(19) United States
(12) Patent Application Publication
(10) Pub. No.: US 2016/0287727 A1
(43) Pub. Date: Oct. 6, 2016

(54) PHOTOACOUSTIC IMAGING CONTRAST AGENT

(71) Applicant: Agency for Science, Technology and Research, Singapore (SG)

(72) Inventors: Malini Olivo, Singapore (SG); Kien Voon Kong, Singapore (SG)

(21) Appl. No.: 15/038,016

(22) PCT Filed: Dec. 9, 2014
(86) PCT No.: PCT/SG2014/000586

§ 371 Date: May 19, 2016

(30) Foreign Application Priority Data
Dec. 9, 2013 (SG) 201309110-3

(51) Int. Cl. A61K 49/22

(52) U.S. Cl. CPC A61K 49/221

(57) ABSTRACT

A photoacoustic imaging contrast agent comprising a biodegradable polymer backbone grafted with a photoacoustic contrast agent is provided. A method of preparing a photoacoustic imaging contrast agent, and a method of imaging living tissue are also provided.

### CROSS-REFERENCE TO RELATED APPLICATION

This application claims the benefit of priority of Singapore patent application No. 201309110-3 filed on 9 Dec. 2013, the content of which is incorporated herein by reference in its entirety for all purposes.

### TECHNICAL FIELD

Various embodiments refer to a photoacoustic imaging contrast agent.

### BACKGROUND

Photoacoustic imaging (PAI) is a rapidly emerging biomedical imaging modality which provides non-invasive, in vivo functional imaging information at clinically relevant penetration depths, while maintaining high spatial resolution and image contrast.

It is a powerful technique which combines optical and ultrasound imaging. It generally involves flashing a laser at low energy onto a target area or region on a subject's body. The laser at low energy may penetrate deeply into the body to create a large radiated area for more detailed imaging. Rapid absorption of laser energy by endogenous chromophores such as hemoglobin and melanin, as well as exogenous contrast agents in tissue may expand the tissue through transient thermo-elastic expansion. This expansion creates ultrasonic acoustic pressure waves that may be detected using ultrasound detectors of appropriate sensitivity, such as ultrasound transducers. The transducer readings may be processed and interpreted using mathematical algorithms to create two dimensional or three dimensional images of the target area to depict the tissue structure. The higher penetration depth of PA imaging (5 cm to 6 cm) over fluorescence and optical coherence tomography (OCT) enables deep tissue imaging, especially in clinical settings.

Researches have been carried out in search for suitable contrast agents to assist in generating images using PAI. For example, endogenous hemoglobin in blood has been used for PA imaging of tumor vascular network in rat brain, blood-oxygenation dynamics in mouse brain, human arm, as well as breast imaging.

This technique may also be coupled with exogenous contrast agents to obtain more information to facilitate accurate diagnoses. Exogenous contrast agents, such as carbon nanotubes (SWNTs), polyhydroxy-fullerene, near-infrared (NIR) dyes like indocyanine green (ICG), as well as gold nanoparticles, have been introduced to enhance imaging contrast. Photoacoustic contrast achieved using the contrast agents, however, may be low, resulting in poor spatial resolution of the images. Clinical application of these contrast agents has also been limited due to cytotoxicity issues.

In view of the above, there remains a need for photoacoustic imaging contrast agents that overcome or at least alleviate one or more of the above-mentioned problems.

### SUMMARY

In a first aspect, a photoacoustic imaging contrast agent comprising a biodegradable polymer backbone grafted with a photoacoustic contrast agent is provided.

In a second aspect, a method of preparing a photoacoustic imaging contrast agent according to the first aspect is provided. The method comprises
a) providing a biodegradable polymer; and
b) grafting a photoacoustic contrast agent to the biodegradable polymer.

In a third aspect, a method of imaging living tissue is provided. The method comprises
a) introducing a photoacoustic imaging contrast agent comprising a biodegradable polymer backbone grafted with a photoacoustic contrast agent into living tissue; and
b) obtaining an image of the living tissue by photoacoustic imaging.

### BRIEF DESCRIPTION OF THE DRAWINGS

The invention will be better understood with reference to the detailed description when considered in conjunction with the non-limiting examples and the accompanying drawings, in which:

FIG. 1 is a schematic diagram depicting synthesis pathway to platinum II-based biodegradable polymeric nanoparticle according to various embodiments.

FIG. 2A to 2E depict characterization of nanoparticles. FIG. 2A is a graph showing molecular weight distribution of [symbol] : poly(AMPD-BAC), and [symbol] : poly(AMPD-BAC) in the incubation with 10 mM glutathione (GSH). Y-axis: abs unit; x-axis: elution volume (mL). FIG. 2B is a graph showing particles size of nanoparticle as monitored by dynamic light scattering (DLS), where [symbol] : poly(AMPD-BAC)-g-PEG, and [symbol] : poly(AMPD-BAC)-g-PEG-DTPA-Pt. Incorporation of the DTPA-Pt has effect on nanoparticle size, in which poly(AMPD-BAC)-g-PEG-DTPA-Pt forms nanoparticle (117.68 nm) with an increase in size of poly(AMPD-BAC)-g-PEG (92 nm). FIG. 2C to 2E are transmission electron microscopy (TEM) images of (C) poly(AMPD-BAC)-g-PEG, (D) poly(AMPD-BAC)-g-PEG-DTPA, and (E) poly(AMPD-BAC)-g-PEG-DTPA-Pt. The spherical morphology of nanoparticle of poly(AMPD-BAC)-g-PEG was affected by incorporation of DTPA due to increase in hydrophilicity of polymer. Spherical nanoparticle was able to reform after complexing with cisplatin, in which the carboxylic acid was bound to cisplatin, which in turn reduced the hydrophilicity of polymer. Scale bar in (C), (D) and (E) denote 100 nm.

FIG. 3A is a graph showing cumulative release of poly(AMPD-BAC)-g-PEG DTPA-Pt in water ( [symbol] ) and in chloride solution (150 mM NaCl) ( [symbol] ). There was no significant release of platinum complex in either water or high chloride solution, which was mainly attributed to the strong chelation of DTPA. FIG. 3B is a graph showing effects of [symbol] poly(AMPD-BAC)-g-PEG, [symbol] poly(AMPD-BAC)-g-PEG-DTPA-Pt, and [symbol] cisplatin on viability of MCF-10A after 24 h incubation. Poly(AMPD-BAC)-g-PEG-DTPA-Pt showed little cytotoxicity to cells as poly(AMPD-BAC)-g-PEG. Cytotoxicity profile of cisplatin was presented for comparison. FIG. 3C to 3E depict optical images of MCF-10A cells 24 h incubation with 10 mg/mL concentrations of (C) Poly(AMPD-BAC)-g-PEG, (D) Poly(AMPD-BAC)-g-PEG-DTPA-Pt, and (E) cisplatin. Scale bar in (C), (D), and (E) denote 100 μm.

FIG. 4A is a graph showing UV-vis spectrum of poly(AMPD-BAC)-g-PEG-DTPA-Pt. FIG. 4B is a graph showing PA signals of a series of different concentration of poly(AMPD-BAC)-g-PEG-DTPA-Pt and water. FIG. 4C are false color images representing relative PA signal strengths of PA B-scan for various concentrations of 15 mg/mL, 10 mg/mL, 8 mg/mL, 5 mg/mL, 1.5 mg/mL, and water (H₂O).

FIG. 5A to 5C show (A) optical image of MCF-10A cells; PA C-scan on MCF-10A (B) before, and (C) after incubation with poly(AMPD-BAC)-g-PEG-DTPA-Pt. Scale bar in (A) is 40 μm. FIG. 5D shows a fluorescence image of MCF-10A cells incubated with FITC (‘FITC labelled’) labeled of poly(AMPD-BAC)-g-PEG-DTPA-Pt. Cell nucleus was stained with DAPI (‘DAPI labelled’). Scale bar in (D) is 30 μm. FIG. 5E are confocal fluorescence images of MCF-10A cells incubated with poly(AMPD-BAC)-g-PEG-DTPA-Pt for 24 h. Images were acquired at different depths of z axis in intervals of 0.5 μm, for −0.5 μm, −1.0 μm, −1.5 μm, −2.0 μm, −2.5 μm, −3.0 μm, −3.5 μm, and −4.0 μm.

FIG. 6A shows PAM imaging of a rat brain in vivo employing poly(AMPD-BAC)-g-PEG-DTPA-Pt and light at a wavelength of poly(AMPD-BAC)-g-PEG-DTPA-Pt (410 nm) nm. The shaded area indicates the selected imaging region. FIG. 6B shows a photograph of the rat cortical blood vessels, with the SSS indicated. FIG. 6C shows PA image acquired before and 20 min after the intravenous injection of poly(AMPD-BAC)-g-PEG-DTPA-Pt. Scale bar in the figure is 1 mm. FIG. 6D shows integrated absorption calculated from the in vivo brain images of six rats at different times following the injection of poly(AMPD-BAC)-g-PEG-DTPA-Pt. The presented values were normalized to that of the integrated absorption of the image obtained before the injection. Y-axis: normalized PA signals (a.u.); x-axis: time (min). FIG. 6E shows in vitro degradation of poly(AMPD-BAC)-g-PEG-DTPA-Pt in a thiols solution mimicking the human plasma thiol composition. [symbol] Tris buffer pH 7.4 and [symbol] solution mimicking human plasma free thiols. Y-axis: cumulative infiltration Pt (%); x-axis: time (min).

FIG. 7 is a schematic diagram showing experimental setup of functional photoacoustic microscopy according to embodiments.

### DETAILED DESCRIPTION

Photoacoustic imaging is based on the mechanism that electromagnetic waveforms, such as radio frequency (rf) or optical waves, may be absorbed by a material, to result in local heating and thermoelastic expansion. The thermoelastic expansion may, in turn, produce megahertz ultrasonic waves in the material, thereby generating a photoacoustic signal. Accordingly, the term “photoacoustic imaging” as used herein refers to signal generation caused by an electromagnetic pulse, with absorption and expansion of a photoacoustic imaging contrast agent, followed by acoustic detection, where the photoacoustic imaging contrast agent absorbs the light energy and converts it to thermal energy that generates the photoacoustic signal.

It has been demonstrated herein that transition metal complexes, despite their complicated chemistry, air- and moisture-sensitivity and toxicity, may be used as photoacoustic contrast agents for grafting to biodegradable polymers to form photoacoustic imaging contrast agents. Advantageously, multiple binding sites may be present on the biodegradable polymer to act as a backbone for grafting the photoacoustic contrast agents, as well as property modifiers such as targeting moieties and/or moieties that modulate bioavailability and/or half-life of the biodegradable polymer. Strong photoacoustic signals have been obtained both in cell imaging and in vivo tests carried out, providing a new platform of photoacoustic imaging contrast agents to cater to specific requirements of preclinical and clinical applications.

With the above in mind, various embodiments refer in a first aspect to a photoacoustic imaging contrast agent. The photoacoustic imaging contrast agent comprises a biodegradable polymer backbone grafted with a photoacoustic contrast agent.

The term “biodegradable polymer” refers to a polymeric material which may be broken down by microorganisms, or which spontaneously breaks down over a relatively short time (within 2-15 months) when exposed to conditions commonly found in nature. For example, a biodegradable polymer may comprise one or more polymeric components that may be completely removed from a localized area by physiological metabolic processes such as resorption.

Examples of biodegradable polymers include, but are not limited to polymers and oligomers of glycolide, lactide, polylactic acid, polyesters of a-hydroxy acids, including lactic acid and glycolic acid, such as the poly(α-hydroxy) acids including polyglycolic acid, poly-DL-lactic, poly-L-lactic acid, and terpolymers of DL-lactide and glycolide; ε-caprolactone and ε-caprolactone copolymerized with polyesters; polylactones and polycaprolactones including poly(ε-caprolactone), poly(δ-valerolactone) and poly (gamma-butyrolactone); polyanhydrides; polyorthoesters; other hydroxy acids; polydioxanone; and other biologically degradable polymers that are non-toxic or are present as metabolites in the body. Examples of polyaminoacids include, but are not limited to, polylysine (PLL), poly L-aspartic acid, poly L-glutamic acid, and styrene-maleic acid anhydride copolymer. Examples of derivatives of polyethylene glycol includes, but are not limited to, poly(ethylene glycol)-di-(ethylphosphatidyl(ethylene glycol)) (PEDGA), poly(ethylene glycol)-co-anhydride, poly(ethylene glycol)co-lactide, poly(ethylene glycol)-co-glycolide and poly (ethylene glycol)-co-orthoester. Examples of acrylamide polymers include, but are not limited to, polyisopropylacrylamide, and polyacrylamide. Examples of acrylate polymers include, but are not limited to, diacrylates such as polyethylene glycol diacrylate (PEGDA), oligoacrylates, methacrylates, dimethacrylates, oligomethoacrylates and PEG-oligoglycolylacrylates. Examples of carboxy alkyl cellulose include, but are not limited to, carboxymethyl cellulose and partially oxidized cellulose.

In various embodiments, the biodegradable polymer backbone is formed by polymerization of a monomer comprising two or more amine groups and a monomer comprising two or more vinyl groups.

The monomer comprising two or more amine groups may be an aminoalkyl piperidine. As used herein, “alkyl” refers to a saturated aliphatic hydrocarbon including straight chain, or branched chain groups, while the term “aminoalkyl” refers to an alkyl group that has been substituted with one or more amino groups. The aminoalkyl group may have 1 to 10 carbon atoms, or may be a medium size alkyl having 1 to 6 carbon atoms such as methyl, ethyl, n-propyl, isopropyl, butyl, iso-butyl, tert-butyl and the like.

Examples of aminoalkyl piperidine include, but are not limited to, 1-methyl-3-(2-aminoethyl)-piperidine, 1-[(2-pyrimidinyl)-aminoalkyl]-piperidine, 2-(1-aminoalkyl) piperidine, 1-(aminoalkyl)piperidine, and mixtures thereof. Specific examples of aminoalkyl piperidine include, but are not limited to, 4-aminomethyl piperidine, 1-(2-chloroethyl)piperidine, N-(3-aminopropyl)piperidine, 1-(2-aminoethyl) piperidine, 1-Methyl, 3-amino piperidine, and mixtures thereof.

In some embodiments, the monomer comprising two or more amine groups is 4-aminomethyl piperidine.

In various embodiments, the monomer comprising two or more vinyl groups is a bisacryl compound. For example, the bisacryl compound may be a bisacrylamide. In some embodiments, the bisacrylamide is N,N′-bis(acryloyl)cystamine.

In various embodiments, the monomer comprising two or more amine groups and/or the monomer comprising two or more vinyl groups contain a disulfide bond. In forming the biodegradable polymer, the biodegradable polymer backbone may also contain a disulfide bond. Advantageously, presence of one or more disulfide bonds in the biodegradable polymer backbone provides enhanced biodegradability, as the one or more disulfide bonds may be easily cleaved into small molecules in the presence of stimuli, such as a reducing agent. The degradation process of disulfide group may take place in a matter of minutes or hours. This is significantly faster than degradation kinetics of other functionalities such as esters and carbonates, where degradation may require days or weeks.

An example of a reducing agent that may be used to cleave disulfide bonds is glutathione, which reduces disulfide bonds by serving as an electron donor. In the process, glutathione may be converted to its oxidized form, glutathione disulfide (GSSG), also called L-(−)-glutathione.

In specific embodiments, the biodegradable polymer backbone is formed by polymerization of N,N′-bis(acryloyl)cystamine with 4-aminomethyl piperidine, which may take place via Michael-addition polymerization. The biodegradable polymer thus formed, poly(aminomethylpiperidine-bis(acryloyl)cystamine) (poly(AMPD-BAC)), is a biodegradable poly(amidoamine) containing multiple reduction-sensitive disulfide bonds as well as secondary and tertiary amines in the backbone. Advantageously, it offers enhanced biodegradability due to its numerous disulfide bonds, which potentially minimizes long term bodily accumulation of contrast agents after photoacoustic imaging is carried out. High density of amino groups in the polymer may also confer significant buffering capacity over a wide pH range, giving rise to the “proton sponge effect” that is important for intracellular delivery. The “proton sponge effect” may arise due to protonation of poly(amidoamine) in a cellular environment. This protonation causes pumping of protons into the endosomes. Following an increase in influx of water into the endosomes, there is swelling and rupturing of the endosomes. This effect based on buffering capacity between pH 7.4 to 6.5 is termed as “proton sponge effect”.

The high density of amino groups in the polymer also provides advantages of easy tunability of their topology, and provides ready availability of multiple sites for attachment of various ligands, such as target-specific ligands and a poly(ethylene)glycol stealth layer, as property modifiers.

In various embodiments, the biodegradable polymer backbone has repeating units of the general formula

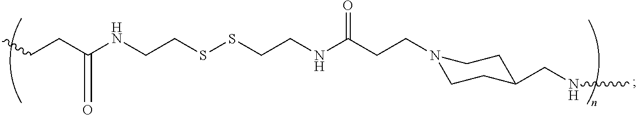

wherein n is an integer in the range of about 1 to 1000.

In various embodiments, n is an integer in the range of about 100 to about 1000, such as about 200 to about 1000, about 400 to about 1000, about 600 to about 1000, about 100 to about 800, about 100 to about 600, about 100 to about 500, about 100 to about 400, about 200 to about 800, or about 400 to about 600.

Depending on the value of n, the biodegradable polymer may have a molecular weight in the range of about 100 Daltons to about 20000 Daltons. For example, the biodegradable polymer may have a molecular weight in the range of about 500 Daltons to about 20000 Daltons, about 1000 Daltons to about 20000 Daltons, about 5000 Daltons to about 20000 Daltons, about 10000 Daltons to about 20000 Daltons, about 15000 Daltons to about 20000 Daltons, about 100 Daltons to about 15000 Daltons, about 100 Daltons to about 10000 Daltons, about 100 Daltons to about 5000 Daltons, about 100 Daltons to about 1000 Daltons, about 1000 Daltons to about 15000 Daltons, about 5000 Daltons to about 15000 Daltons, about 10000 Daltons to about 15000 Daltons, or about 5000 Daltons to about 10000 Daltons.

As mentioned above, the biodegradable polymer backbone is grafted with a photoacoustic contrast agent. As used herein, the term “photoacoustic contrast agent” is used interchangeably with the term “optoacoustic contrast agent”, and refers generally to a compound used to enhance contrast of living tissue, to thereby improve visibility thereof when acquiring optical images by photoacoustic imaging. A contrast agent may be helpful in qualitatively and quantitatively determining disease and/or injury by improving visibility and contrast of an object of interest, such as living tissue, or internal body structures such as vessels or organs.

The term “graft” as used herein refers to chemical attachment of a compound and/or a moiety onto another material. For example, the photoacoustic contrast agent may be grafted to the biodegradable polymer backbone by covalent bonding. The photoacoustic contrast agent may contain one or more functional groups to allow covalent bonding with one or more functional groups on the biodegradable polymer backbone, so as to graft the photoacoustic contrast agent to the biodegradable polymer backbone. Examples of functional group include, but are not limited to, a carboxyl group, a hydroxyl group, a nitro group, a halogen group, a cyano group, an epoxy group, an organophosphorous group, and an amine group. In various embodiments, the photoacoustic contrast agent is able to graft directly to the biodegradable polymer backbone, i.e. a cross-linking agent is not required.

Examples of photoacoustic contrast agent include a transition metal, carbon nanomaterials such as carbon nanotubes, fullerene and graphene, near-infrared (NIR) dyes such as indocyanine green (ICG), and gold nanoparticles.

In specific embodiments, the photoacoustic contrast agent is a transition metal complex. As used herein, the term “transition metal complex” refers to a compound containing a transition metal which is linked to one or more ligands. The term “ligand” refers to atoms or groups of atoms, which forms coordination bonds to the transition metal atom. The ligands may have different ligations, such as monodentate, bidentate, tridentate, and tetradentate.

The transition metal complex may have the general formula -L′-M, wherein L¹ is a chelating ligand and M is a transition metal.

As used herein, the term “chelating ligand” refers to a compound which is able to form coordinated bonds with metal ions through two or more of its atoms. A heterocyclic ring including the metal ions may be formed as a result.

In various embodiments, the chelating ligand L¹ is selected from the group consisting of an aminopolycarboxylic acid, a succinimidyl ester, an isothiocyanate, derivatives thereof, and combinations thereof.

In some embodiments, the chelating ligand L′ is selected from the group consisting of diethylene triamine pentaacetic acid, COOH-PEG-succinimidyl esters, succinimidyl esters-amino acid, succinimidyl esters-PEG-SH, COOH-PEG-isothiocyanates, isothiocyanates-amino acid, isothiocyanates-PEG-SH, derivatives thereof, and combinations thereof.

The term “transition metal” as used herein may refer to a metal in Group 3 to 12 of the Periodic Table of Elements, such as titanium (Ti), vanadium (V), niobium (Nb), tantalum (Ta), chromium (Cr), molybdenum (Mo), tungsten (W), manganese (Mn), iron (Fe), ruthenium (Ru), osmium (Os), iridium (Ir), nickel (Ni), copper (Cu), technetium (Tc), rhenium (Re), cobalt (Co), rhodium (Rh), iridium (Ir), palladium (Pd), platinum (Pt), silver (Ag), gold (Au), a lanthanide such as europium (Eu), gadolinium (Gd), lanthanum (La), ytterbium (Yb), and erbium (Er), or a post-transition metal such as gallium (Ga), and indium (In).

In various embodiments, the transition metal is a metal selected from Group 3 to 12 of the Periodic Table of Elements. In various embodiments, the transition metal is Pt (II). The free ligand binding sites of the transition metal may be occupied by any suitable ligands such as NH₃.

In some embodiments, M may be cis-diamine platinum (II) having formula Pt(NH3)₂.

In addition to the photoacoustic contrast agent, the biodegradable polymer backbone may be further grafted with at least one of a targeting moiety or a moiety that modulates at least one of bioavailability or half-life of the biodegradable polymer.

Targeting ability of the photoacoustic imaging contrast agent may be achieved by incorporating a variety of targeting moieties.

In various embodiments, the targeting moiety may have general formula -L²-P, wherein L² is a bond or a linker, and P is selected from the group consisting of a polyalkylene glycol, folic acid, an antibody, a targeting protein, and a targeting peptide.

L² may be a bond, or a linker that allows grafting of the targeting moiety to the biodegradable polymer backbone. Depending on P and the biodegradable polymer backbone, different L² may be used. For example, L² may be an optionally substituted C₁-C₂₀ alkyl or 2-20 membered heteroalkyl, an optionally substituted monocyclic, condensed polycyclic or bridged polycyclic C₅-C₂₀ aryl or 5-20-membered heteroaryl, hydroxy, alkoxy, cyano, halogen, nitro, silyl, amino, or succinimidyl ester, to name only a few. In some embodiments, L² is succinimidyl ester.

P is selected from the group consisting of a polyalkylene glycol, folic acid, an antibody, a targeting protein, and a targeting peptide.

For instance, P may be an antibody, for example a monoclonal or polyclonal antibody, which immunologically binds to a target analyte at a specific determinant or epitope. The term “antibody” is used in the broadest sense and specifically covers monoclonal antibodies as well as antibody variants, fragments or antibody like molecules, such as for example, Fab, F(ab′)₂, scFv, Fv diabodies and linear antibodies, so long as they exhibit the desired binding activity.

In some embodiments, P is a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. The monoclonal antibodies may include “chimeric” antibodies and humanized antibodies. A “chimeric” antibody is a molecule in which different portions are derived from different animal species, such as those having a variable region derived from a murine mAb and a human immunoglobulin constant region.

In some embodiments, P is a polyclonal antibody. “Polyclonal antibodies” refer to heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate optionally supplemented with adjuvants.

“Peptide” generally refers to a short chain of amino acids linked by peptide bonds. Typically peptides comprise amino acid chains of about 2-100, more typically about 4-50, and most commonly about 6-20 amino acids. “Polypeptide” generally refers to individual straight or branched chain sequences of amino acids that are typically longer than peptides. “Polypeptides” usually comprise at least about 20 to 1000 amino acids in length, more typically at least about 100 to 600 amino acids, and frequently at least about 200 to about 500 amino acids. Included are homo-polymers of one specific amino acid, such as for example, poly-lysine. “Proteins” include single polypeptides as well as complexes of multiple polypeptide chains, which may be the same or different.

Multiple chains in a protein may be characterized by secondary, tertiary and quaternary structure as well as the primary amino acid sequence structure, may be held together, for example, by disulfide bonds, and may include post-synthetic modifications such as, without limitation, glycosylation, phosphorylation, truncations or other processing.

Antibodies such as IgG proteins, for example, are typically comprised of four polypeptide chains (i.e., two heavy and two light chains) that are held together by disulfide bonds. Furthermore, proteins may include additional components such associated metals (e. g., iron, copper and sulfur), or other moieties. The definitions of peptides, polypeptides and proteins includes, without limitation, biologically active and inactive forms; denatured and native forms; as well as variant, modified, truncated, hybrid, and chimeric forms thereof.

The terms “analyte”, “target compound”, “target molecule” or “target” as interchangeably used herein, refer to any substance that may be detected in an assay by binding to a binding molecule, and which, in embodiments, may be present in a sample. Therefore, the analyte may be, without limitation, any substance for which there exists a naturally occurring antibody or for which an antibody can be prepared. The analyte may, for example, be an antigen, a protein, a polypeptide, a nucleic acid, a hapten, a carbohydrate, a lipid, a cell or any other of a wide variety of biological or non-biological molecules, complexes or combinations thereof. Generally, the analyte will be a protein, peptide, carbohydrate or lipid derived from a biological source such as bacterial, fungal, viral, plant or animal samples. Additionally, however, the target may also be a small organic compound such as a drug, drug-metabolite, dye or other small molecule present in the sample.

The term “sample”, as used herein, refers to an aliquot of material, frequently biological matrices, an aqueous solution or an aqueous suspension derived from biological material. Samples to be assayed for the presence of an analyte include, for example, cells, tissues, homogenates, lysates, extracts, and purified or partially purified proteins and other biological molecules and mixtures thereof.

Non-limiting examples of samples include human and animal body fluids such as whole blood, serum, plasma, cerebrospinal fluid, sputum, bronchial washing, bronchial aspirates, urine, semen, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed. The samples used may vary based on the assay format and the nature of the tissues, cells, extracts or other materials, especially biological materials, to be assayed. Methods for preparing protein extracts from cells or samples are well known in the art and can be readily adapted in order to obtain a sample that may be used with the photoacoustic imaging contrast agents disclosed herein. Detection in a body fluid can also be in vivo, i.e. without first collecting a sample.

Other specific examples of targeting moieties include RGD peptides for targeting cellular adhesion molecules such as in vasculature endothelial cells in solid tumors; NGR peptides for targeting aminopeptidase N (CD13) such as in vasculature endothelial cells in sold tumors; folate for targeting folate receptor such as in cancer cells that overexpress the folate receptors; transferrin for targeting transferrin receptor such as in cancer cells that overexpress the transferrin receptors; GM-CSF for targeting GM-CSF receptor such as in leukaemic blasts; glactosamine for targeting galactosamine receptor on hepatocytes such as in hepatoma; anti-EGFR for targeting vasculature endothelial growth factor receptor such as in vasculature endothelial cells in solid tumours; anti-ERBB2 for targeting ERBB2 receptor such as in cells that overexpress the ERBB2 receptor, for example in breast and ovarian cancers; anti-CD20 for targeting CD20, a B-cell surface antigen such as in non-Hodgkin's lymphoma and other B-cell lymphoproliferative diseases; and anti-MUD for targeting MUC1, an aberrantly such as in breast and bladder cancer.

The biodegradable polymer backbone may also be grafted with a moiety that modulates at least one of bioavailability or half-life of the biodegradable polymer.

For example, the moiety that modulates at least one of bioavailability or half-life of the biodegradable polymer may reduce non-specific adsorption of proteins, to allow longer durations of in vivo circulation, which may in turn improve delivery efficacy.

In various embodiments, the moiety that modulates at least one of bioavailability or half-life of the biodegradable polymer may be a polyalkylene glycol. Examples of polyalkylene glycol include polyethylene glycol, polypropylene glycol, polybutylene glycol, polypentylene glycol, polyhexylene glycol, polyheptylene glycol, polyoctylene glycol, polynonylene glycol, polydecylene glycol, branched and structural isomers thereof.

In some embodiments, the polyalkylene glycol comprises or consists of polyethylene glycol. The polyethylene glycol may be activated with a 4-nitrophenyl carbonate group, which allows ready reaction of secondary amines that may be present on the polymer backbone to graft the polyethylene glycol on the polymer backbone.

The photoacoustic contrast agent, the targeting moiety, and/or the moiety that modulates at least one of bioavailability or half-life of the biodegradable polymer may each be coupled to a secondary amine group in the polymer backbone.

For example, the photoacoustic imaging contrast agent may have repeating units of the general formula

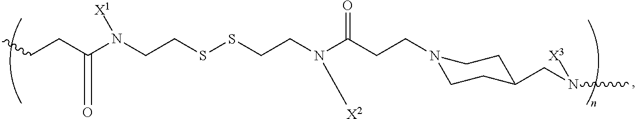

wherein X¹, X², and X³ at each occurrence is independently selected from the group consisting of H, a photoacoustic contrast agent, a targeting moiety, and a moiety that modulates at least one of bioavailability or half-life of the biodegradable polymer, wherein at least one occurrence of at least one of X¹, X², or X³ is a photoacoustic contrast agent, and n is an integer in the range of about 1 to about 1000.

In specific embodiments, the photoacoustic imaging contrast agent has the general formula

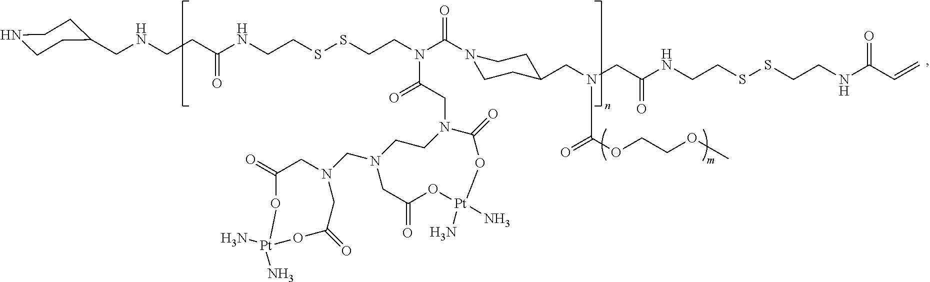

wherein in is an integer in the range of about 1 to 10 and n is an integer in the range of about 1 to about 1000.

The photoacoustic imaging contrast agent may be in the form of a nanoparticle. A “nanoparticle” refers to a particle having a characteristic length, such as diameter, in the range from 1 and 500 nanometers, such as 10, 20, 30, 40, 50, 60, 70, 80, 90, 100, 150, 200, 400, or 500 nm.

The nanoparticle may be irregular or regular in shape. In some embodiments, the nanoparticle is regular in shape, such as a nanosphere. Size of the nanoparticle may be characterized by its diameter. The term “diameter” as used herein refers to the maximal length of a straight line segment passing through the center of a figure and terminating at the periphery. Although the term “diameter” is used normally to refer to the maximal length of a line segment passing through the centre and connecting two points on the periphery of a nanosphere, it is also used herein to refer to the maximal length of a line segment passing through the centre and connecting two points on the periphery of nanoparticles having other shapes, such as a irregularly shaped nanoparticle. In embodiments where more than one nanoparticle is present, size of the nanoparticles may be characterized by their mean diameter, wherein the term “mean diameter” refers to an average diameter of the nanoparticles, and may be calculated by dividing the sum of the diameter of each nanoparticle by the total number of nanoparticles.

In various embodiments, the photoacoustic imaging contrast agent has a size in the range of about 80 nm to about 150 nm, such as about 80 nm to about 120 nm, or about 90 nm to about 120 nm. Advantageously, photoacoustic imaging contrast agents disclosed herein may have a size that allows optimal blood circulation time, and hence are particularly suitable for blood vascular imaging.

Various embodiments refer in a second aspect to a method of preparing a photoacoustic imaging contrast agent comprising a biodegradable polymer backbone grafted with a photoacoustic contrast agent. The method includes providing a biodegradable polymer; and grafting a photoacoustic contrast agent to the biodegradable polymer.

Examples of biodegradable polymer and photoacoustic contrast agent that may be used have already been described above.

In various embodiments, the photoacoustic contrast agent is a transition metal complex. For example, the transition metal complex may have the general formula wherein L¹ is a chelating ligand, and M is a transition metal.

In some embodiments, grafting a transition metal complex to the biodegradable polymer comprises adding a chelating agent to the biodegradable polymer to graft the chelating agent to the biodegradable polymer, with subsequent addition of a compound containing transition metal, so that the chelating agent forms coordinated bonds with the transition metal ions through two or more of its atoms, to form a transition metal complex. In so doing, the transition metal complex thus formed is grafted to the biodegradable polymer.

In specific embodiments, the compound containing transition metal is cisplatin. Cisplatin is a platinum complex having formula PtCl₂(NH₃)₂, and has been heralded as one of the most effective chemotherapy agents approved by the US Food and Drug Administration (FDA). As demonstrated herein in the examples, Cisplatin may be used in a method of preparing a photoacoustic imaging contrast agent comprising a biodegradable polymer backbone grafted with a photoacoustic contrast agent.

Grafting a photoacoustic contrast agent to the biodegradable polymer may include grafting a targeting moiety and/or a moiety that modulates at least one of bioavailability or half-life of the biodegradable polymer to the biodegradable polymer. Examples of targeting moieties and moieties that modulate at least one of bioavailability or half-life of the biodegradable polymer have already been discussed above. Grafting of each of the photoacoustic contrast agent, the targeting moiety, and/or the moiety that modulates at least one of bioavailability or half-life of the biodegradable polymer may take place via a secondary amine group in the polymer backbone.

Various embodiments refer in a third aspect to method of imaging living tissue. The method includes introducing a photoacoustic imaging contrast agent comprising a biodegradable polymer backbone grafted with a photoacoustic contrast agent into living tissue; and obtaining an image of the living tissue by photoacoustic imaging.

The photoacoustic imaging may be a laser-based photoacoustic imaging. The photoacoustic imaging may be carried out at a wavelength in the range of about 400 inn to about 900 nm, which correspond to the visible range (400 nm to 750 nm) and near infrared region of the spectrum (750 nm to 900 nm).

Obtaining an image of the living tissue may be carried out in vivo or in vitro. In some embodiments, obtaining an image of the living tissue is carried out in vivo. In other embodiments, the living tissue is contained in a sample, and obtaining an image of the living tissue is carried out in vitro.

In various embodiments, the photoacoustic imaging is photoacoustic microscopy. For example, the photoacoustic microscopy may include laser pulse generation, delivery of the laser pulse to a subject under study, reception of photoacoustic signal generated from the subject, image reconstruction, and display of the image. Exemplary illustrations of how photoacoustic microscopy may be carried out are provided in the examples disclosed herein.

Hereinafter, the present invention will be described more fully with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, lengths and sizes of layers and regions may be exaggerated for clarity.

As used herein, the term “and/or” includes any and all combinations of one or more of the associated listed items. The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Photoacoustic microscopy (PAM) is an emerging imaging diagnostic technique for various diseases. Coupled with contrast agents, photoacoustic (PA) imaging yields additional information to facilitate an accurate diagnosis.

Herein, a novel platinum II-based biodegradable nanoparticle that shows significant promise as a PA contrast agent is presented, and its performance in improving sensitivity for in vivo brain vascular imaging in rat has been demonstrated. The platinum II-based biodegradable nanoparticle for PA imaging is capable of effective cellular internalization with very low cytotoxicity. To the best of the inventors' knowledge, this is the first reported preparation of PA contrast agents using a biodegradable polymer.

The experiment results show great promise as a novel photoacoustic contrast agent as its strong PA signal was observed both in cell imaging and in vivo rat cerebral vascular imaging via designed PAM. This work exemplifies the incorporation of transition metal complex with polymeric nanoparticles, further expanding the field of the ability of PA imaging. As such, it is in line with current interests in nanoformulation to facilitate the development of novel contrast agents for highly accurate diagnosis. The successful demonstration of PA imaging with biodegradable polymer may benefit currently delivery processes of PA agents. Also, the inventors' believe that this work illustrates the applicability of using polymeric nanoparticle functionalized with other transition metals in the development of PA contrast agents.

### Example 1

### Materials

4-aminomethyl piperidine (AMPD, Alfa Aesar), N,N′-bis(acryloyl)cystamine (BAC, polysciences), paclitaxel (PTX, Yunnan Hande Bio-Tech Co. Ltd, China), dithiothreitol (DTT, Sigm-Aldrich), 4-nitrophenyl chloroformate (Fluka), cisplatin (Sigma), diethylenetriaminepentaacetic acid dianhydride (Sigma) and monomethyl poly(ethylene glycol) (Mw: 2000, Sigma-Aldrich) were from commercial sources and used without further purification.

All other starting materials were purchased from Aldrich. MDA-MB-231 cell lines were purchased from the American Type Culture Collection (ATCC). MDA-MB-231 cells were grown in culture flasks with Dulbecco's Modified Engle Medium (DMEM, Invitrogen) containing 10% fetal bovine serum (FBS, Invitrogen), 1% L-glutamate (GIBCO Laboratories) and 1% penicillin-streptomycin (GIBCO Laboratories) at 37° C. in a 5% CO2 incubator. Phosphate buffered saline (PBS) was purchased from 1st BASE. The cyctotoxicity assay were performed with 3-(4,5-Dimethylthiazol-2-yl)-2,5-Diphenyltetrazolium Bromide (MTT, Duchefa Biochemie).

### Example 2

### Characterization

¹H NMR spectra were recorded on a Bruker DRX-400 NMR spectrometer; chemical shifts reported were referenced against the residual proton signals of the solvents.

Molecular weight was determined from gel permeation chromatography (GPC) implemented on a Waters 2690 apparatus with Water Ultrahydrogel 250 and 200 columns, a Waters 410 refractive index detector, and a miniDAWN light scattering detector (Wyatt Technology) using 0.2M acetic acid/0.2M sodium acetate as eluent at a flow rate of 0.75 ml/min.

A Brookhaven BI-9000AT Digital Autocorrelator was used for dynamic light scattering (DLS) measurements with 90° scattering angle laser light of wavelength 632.8 nm. Isocratic reverse phase high performance liquid chromatography (HPLC) was implemented on the Waters 2695 Separation Module with a reverse phase SymmetryShield® Column (pore size 5 μm, 150 mm×4.6 mm i.d.) and a Waters 2996 PDA detector with Millennium processing software version 3.2. A mixture solvent of acetonitrile-water (50:50, v/v) was used as mobile phase at a flow rate of 1 ml/min at 25° C., and the wavelength of the UV detector was set at 227 nm.

Transmission electron microscopy (TEM) images were obtained on a Philips CM300 FEGTEM instrument at 300 kV. The samples were prepared by dipping holey copper meshes covered with carbon into an aqueous solution of samples followed by drying in air.

### Example 3

### Synthesis of Linear Polymer Poly(AMPD-BAC)

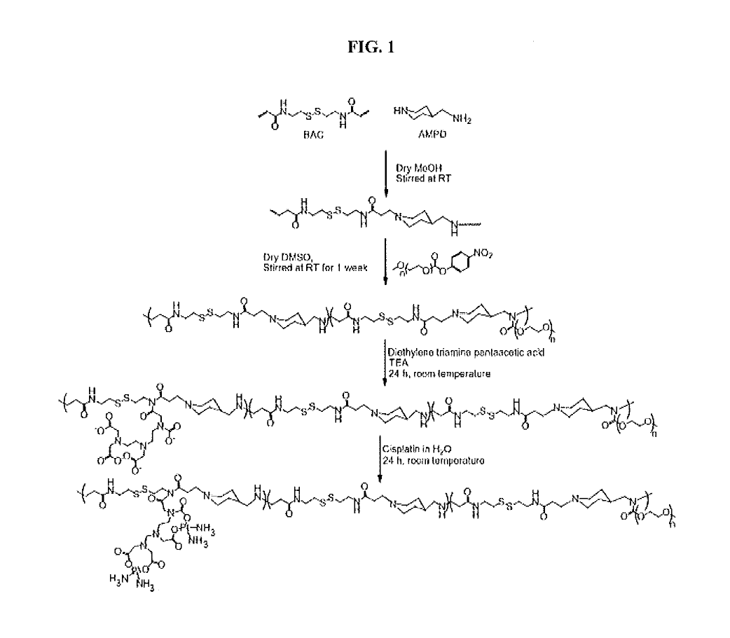

FIG. 1 is a schematic diagram showing synthesis of biodegradable polymeric nanoparticle tethered platinum (II). The polymer was synthesized by Michael-addition polymerization of triamine of 4-aminomethyl piperidine (AMPD) with an equimolar bisacrylamide (BAC) in methanol.

Specifically, BAC (6.14 g, 25 mmol) was dissolved in 40 ml dry methanol at room temperature. AMPD (2.88 g, 25 mmol) was added to the solution while stirring. The mixture was stirred at room temperature for 30 days. The resulted solution was dialyzed against methanol for five times to remove unreacted monomers.

Yield=6.80 g (71%). ¹H NMR (CH₃OD): δ 1.24 (m, 2H, CH₂), 1.52 (bs, 1H, CH), 1.75 (d, 2H, CH₂), 2.03 (t, 4H, CH₂), 2.38 (t, 4H, CH₂), 2.49 (d, 2H, CH₂), 2.65 (t, 2H, CH₂), 2.83 (t, 4H, CH₂), 2.95 (d, 2H, CH₂), 3.49 (t, 4H, CH₂).

### Example 4

### Synthesis of poly(AMPD-BAC)-g-PEG

PEG 4-nitrophenyl carbonate (5.83 g, 2.6 mmol) was added to poly(AMPD-BAC) (6.0 g, 16.0 mmol) in 60 ml dry dimethylsulfone. The mixture was stirred at room temperature for five days. The resulted solution was dialyzed against methanol for 4 times to remove unreacted PEG. The solvent was removed under reduced pressure to afford poly(AMPD-BAC)-g-PEG as a water soluble white solid.

Yield=7.40 g (73%). ¹H NMR (CH₃OD): δ 1.24 (m, 2H, CH₂), 1.52 (bs, 1H, CH), 1.75 (d, 2H, CH₂), 2.03 (t, 4H, CH₂), 2.38 (t, 4H, CH₂), 2.49 (d, 211, CH₂), 2.65 (t, 2H, CH₂), 2.83 (t, 4H, CH₂), 2.95 (d, 2H, CH₂), 3.49 (t, 4H, CH₂), 3.63 (s, b, OCH₂CH₂), 4.14 (bs, 2H, CH₂).

### Example 5

### Synthesis of poly(AMPD-BAC)-g-PEG-DTPA

Diethylenetriaminepentaacetic acid dianhydride (0.74 g, 2.1 mmol) and distilled triethylamine (0.3 ml, 2.1 mmol) to a poly(AMPD-BAC)-g-PEG (2.0 g, 0.21 mmol) in 30 ml dry DMSO. After the mixture was stirred for 24 h under argon at room temperature, the resulted solution was dialyzed against water for five times and then freeze dried to afford poly(AMPD-BAC)-g-PEG-DTPA which is a white solid.

Yield=1.0 g (50%). ¹H NMR (CDCl3): δ 1.24 (m, 2H, CH₂), 1.52 (bs, 1H, CH), 1.75 (d, 2H, CH₂), 2.03 (t, 4H, CH₂), 2.38 (1, 4H, CH₂), 2.49 (d, 2H, CH₂), 2.65 (t, 2H, CH₂), 2.83 (t, 4H, CH₂), 2.95 (d, 2H, CH₂), 3.49 (t, 4H, CH₂), 3.51 (s, DTPA), 3.78 (s, DTPA), 3.83 (s, DTPA), 3.96 (s, 2H, CH₂ DTPA), 3.63 (s, b, OCH₂CH₂), 4.14 (bs, 2H, CH₂).

### Example 6

### Preparation of Cisplatin Incorporated Micelles

Poly(AMPD-BAC)-g-PEG-DTPA (1 g, 0.1 mmol) was dissolved in deionised water. To this solution, cisplatin (0.2 g, 0.7 mmol) was added. pH of the solution was adjusted to pH 7 with 0.1 M NaOH, and stirred for 72 h. The resulted solution was dialyzed against water for five times (MWCO: 2000) to remove unbound cisplatin, and then freeze dried to afford an orange solid. Final concentration of platinum was measured by ICP-MS.

### Example 7

### Preparation of FITC labelled poly(AMPD-BAC)-g-PEG-DTPA-Pt

FITC was attached to poly(AMPD-BAC)-g-PEG-DTPA via reaction among isothiocyanate groups of FITC and the residual amines on poly(AMPD-BAC)-g-PEG-DTPA. In a typical process, 0.1 g of poly(AMPD-BAC)-g-PEG-DTPA was dissolved in 4 mL of dried DMSO. Then a solution of 70 mg of FITC in 2 mL of DMSO was added dropwise. The reaction was performed in dark at ambient temperature overnight. The solution was dialyzed in methanol for five times.

### Example 8

### Platinum In-Vitro Release Study

Poly(AMPD-BAC)-g-PEG-DTPA-Pt prepared was transferred into a dialysis bag (MWCO:2000). After that, the dialysis bag was placed into a conical flask containing 40 mL of phosphate buffered saline and 150 mM NaCl. The conical flask was kept in 37.4° C. water bath and was being stirred at 100 rpm throughout the release study. The release was sampled at a defined time period, and measured by ICP-MS.

### Example 9

### Cytotoxicity Assay

MDA-MB-231 cells were cultured in DMEM supplemented with 10% FBS at 37° C., 10% CO₂, and 95% relative humidity.

For cell viability assay, polymer solutions were prepared in serum supplemented tissue culture medium. The cells (10,000 cells/well) were seeded into 96-well microtiter plates (Nunc, Wiesbaden, Germany). After overnight incubation, the culture medium was replaced with 100 μL of serial dilutions of the polymers, and the cells were incubated for 24 and 48 hours. 20 μL of sterile filtered MTT (3-(4,5-dimethylthiazol-2-yl)-2,5-diphenyl tetrazolium bromide) (5 mg mL⁻¹) stock solution in phosphate buffered saline (PBS) was added to each well. After 4 hours, unreacted dye was removed by aspiration. The formazan crystals were dissolved in 100 μL/well DMSO (BDH laboratory Supplies, England) and measured spectrophotometrically in an ELISA reader (Model 550, Bio-Rad) at a wavelength of 570 nm. The spectrophotometer was calibrated to zero absorbance using culture medium without cells. The relative cell growth (%) related to control cells containing cell culture medium without polymer was calculated by [A]test/[A]control×100%. All the tests were performed in triplicate.

### Example 10

### Fluorescence Labelling

Cells were grown on coverslips (60% confluence), incubated with solution of FITC labeled poly(AMPD-BAC)-g-PEG-DTPA-Pt for 24 h, washed with 1×PBS (3×2 ml), fixed with 5% formaldehyde in PBS for 20 min at room temperature. They were then permeabilized with 0.1% Triton X-10 in PBS (USB Corp). The sample was then washed with 1×PBS (3×3 ml), mounted with 200 μl DAPI containing ProLong Gold antifade reagent (Invitrogen). Laser confocal fluorescence micrographs were obtained on Leica TCS SP5X. For DAPI imaging, the emission was observed at 421 nm with an excitation at 401 nm; and for FITC imaging, the emission was observed at 517 nm with an excitation 495 nm.

### Example 11

### Dark Field Confocal Photoacoustic Microscopy System

The 50-MHz dark-field confocal PAM system used for imaging vascular in the rat brain is shown in figure. The system includes laser pulse generation and delivery, PA signal reception, and image reconstruction and display.

Laser pulses, 4 ns wide, were generated at a frequency of 10 Hz by using an optical parametric oscillator (Surlite OPO Plus, Continuum, USA). The laser was pumped by a frequency-tripled Nd:YAG Q-switched laser (Surlite II-10, Continuum, USA). At the selected wavelengths, the designed contrast agent is a dominant optical absorber, producing strong optical absorption and thus guaranteeing that the detected PA signals mainly come from contrast agent. The acquired PA signal at λ₄₁₀ₙₘ is sensitive to changes in Os salt contrast agent. The 50-MHz ultrasonic transducer used in the current PAM system was custom-made by the Acoustic Sensor Co., Ltd at Taiwan. It has a −6 dB fractional bandwidth of 57.5%, a focal length of 9 mm and a 6 mm active element, offering an axial resolution of 32 μm and a lateral resolution of 61 μm.

Laser energy was delivered using a 1 mm multimodal fiber. The fiber tip was coaxially aligned with a convex lens, an axicon, a plexiglass mirror, and an ultrasonic transducer on an optical bench, forming dark-field illumination that was confocal with the focal point of the ultrasonic transducer. Incident energy density on the sample surface was well within American National Standards institute (ANSI) safety limits. The transducer was immersed in an acrylic water tank during the imaging process, and the hole at the bottom of the tank was sealed with a piece of 15 pin thick polyethylene film.

A thin layer of ultrasonic gel was applied as a PA conductive medium, which was then attached to the thin polyethylene film to ensure reliable coupling of the PA waves with the water tank. The PA signals received by the ultrasonic transducer were pre-amplified by a low-noise amplifier (noise figure 1.2 dB, gain 55 dB, AU-3A-0110, USA), cascaded to an ultrasonic receiver (5073 PR, Olympus, USA) and then digitized and sampled by a computer-based 14-bit analog to digital (A/D) card (CompuScope 14220, GaGe, USA) at a 200-MHz sampling rate for data storage.

Fluctuations in the laser energy were monitored with a photodiode (DET36A/M, Thorlabs, USA). The recorded photodiode signals were measured prior to the experiment to compensate for PA signal variations caused by laser-energy instability. The achievable penetration depth of the current PA microscopy system was 3 mm with approximately 18-dB SNR, where SNR is defined as the ratio of the signal peak value to the root-mean-square value of the noise. Three scan types can be provided by this system: A-line (i.e., one-dimensional images where the axis represents the imaging depth), B-scan (i.e., two-dimensional images where one axis is the lateral scanning distance and the other is the imaging depth), and C-scan (i.e., projection images from the three-dimensional images). The amplitude of the envelope-detected PA signals was used in the subsequent functional imaging analysis.

For in vitro testing, the PA contrast of the designed agent, polyethylene tubing (˜20 cm) was filled with various designed samples. Afterwards, the tubing was positioned at a depth of the transducer's focus, i.e., depth of 9 mm with respect to the transducer in water tank. The system was maintained in a 25° C. water bath throughout the experiment. The contrast changes of the developed probes were imaged using the designed PA microscopy system with 32×61-μm resolution. The scanning step size was 20 μm for each B-scan of in vivo and in vitro experiments.

### Example 12

### Experimental Animals

Six male Wistar rats (NUS-CARE, Singapore), weighing 250 g to 300 g, were used. The animals were housed at a constant temperature and humidity with free access to food and water. Before imaging experiments, the rats fasted for 24 hours but were given water ad libitum. All animal experiments were conducted in accordance with guidelines from Institutional Animal Care and Use Committee (IACUC) at the National University of Singapore.

Rats remained anesthetized with isoflurane 2% to 3% in 100% O₂ and were mounted on a dorsal position over a custom-made acrylic stereotaxic holder. Anesthetized rats were mounted on the custom-made acrylic stereotaxic head holder and the skin and muscle were cut away from the skull to expose the bregma landmark. The anteroposterior (AP) distance between the bregma and the interaural line was directly surveyed. The bregma was 9.3±0.12 mm (mean±standard deviation [SD]) anterior to the interaural line.

Furthermore, a craniotomy was performed for each animal, and a bilateral cranial window of approximately 4 (horizontal)×3 (vertical) mm size was made with a high-speed drill. After the rat was secured to the stereotaxic frame and placed on the bed pallet, the pallet was moved to position at the bregma, which was 9 mm anterior to an imaginary line drawn between the centers of each ear bar (the interaural line). The interaural and bregma references were then used to position the heads in the PAM system, without additional surgery, in the following experiments.

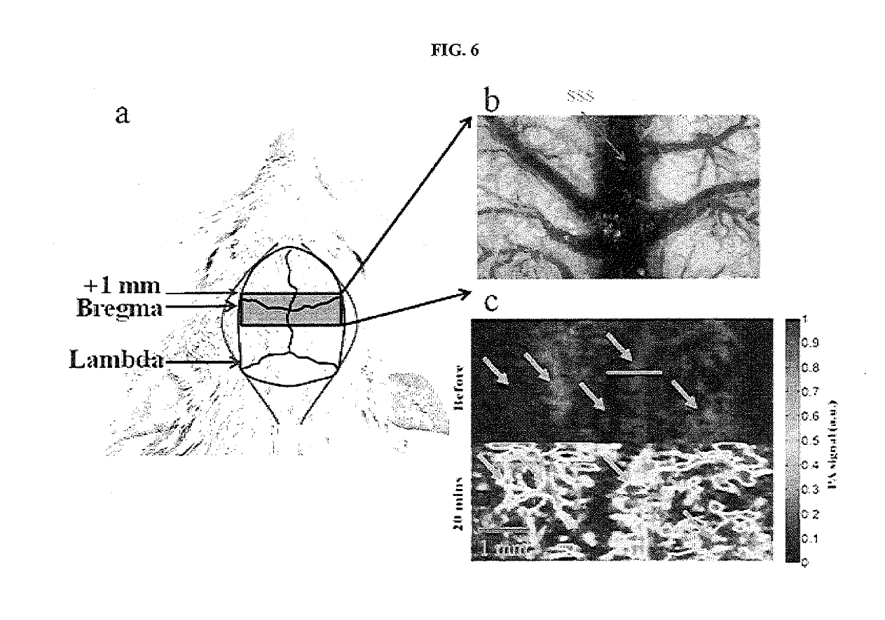

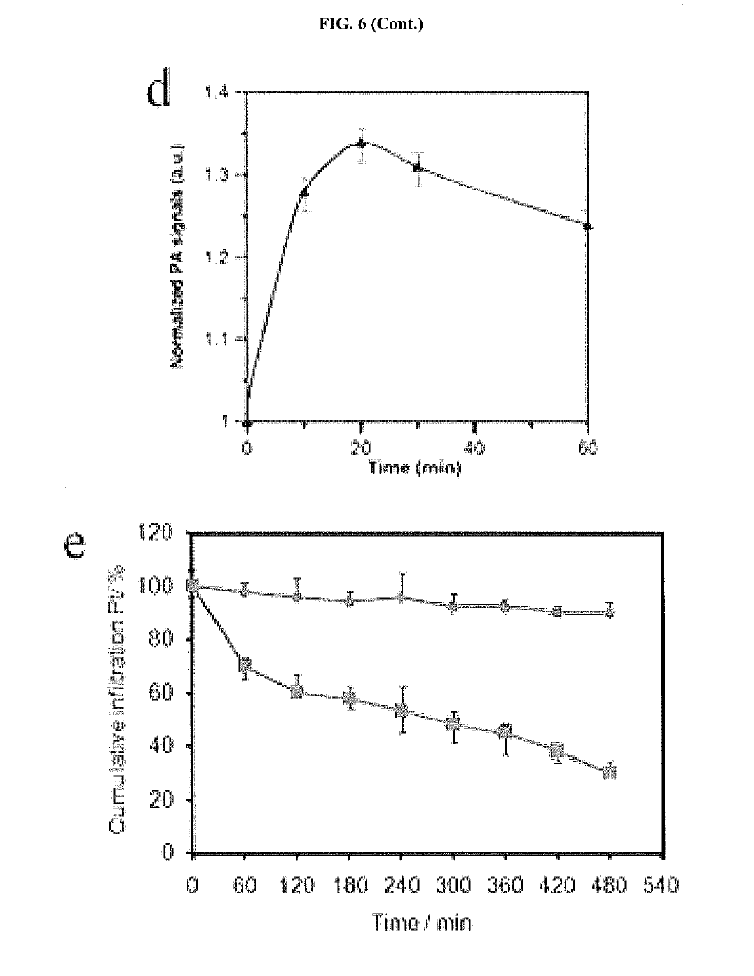

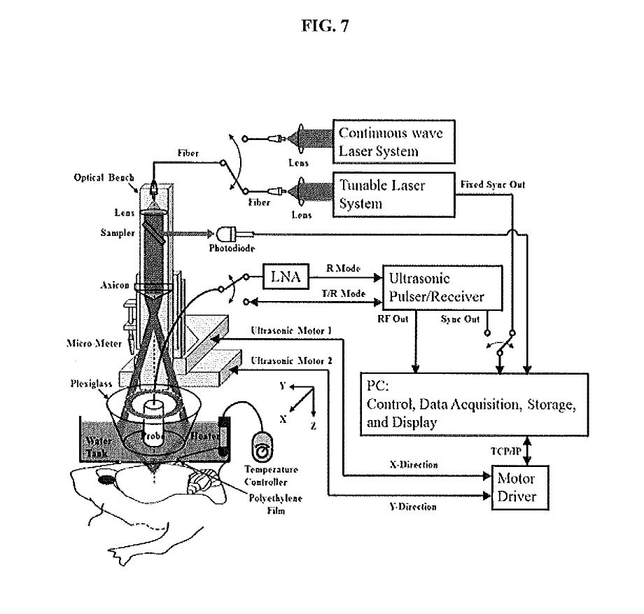

After bregma positioning, a PA C-scan (i.e., projection image from the three-dimensional images) was performed to acquire reference images of the cortical vasculature. The cortical blood vessels under the open-skull window of the rat cortical surface (as shown in FIG. 6A), were imaged in vivo by PAM at λ₄₁₀, as shown in FIG. 6C. The SSS is the largest vein in the rodent brain cortex and is the region-of-interest (ROI) in this study. The ROI cross-section location is indicated in FIG. 6C by a solid line.

### Example 13

### Results and Discussion

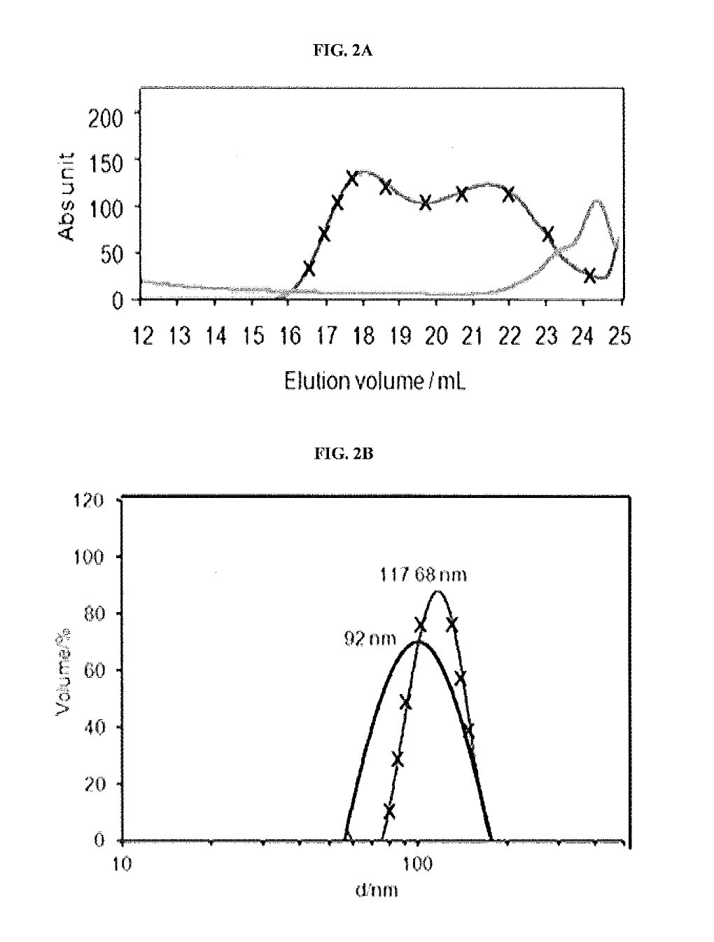

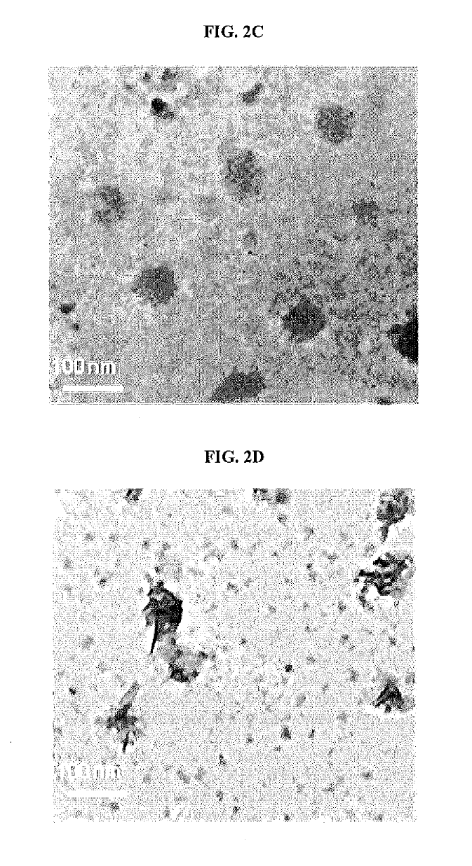

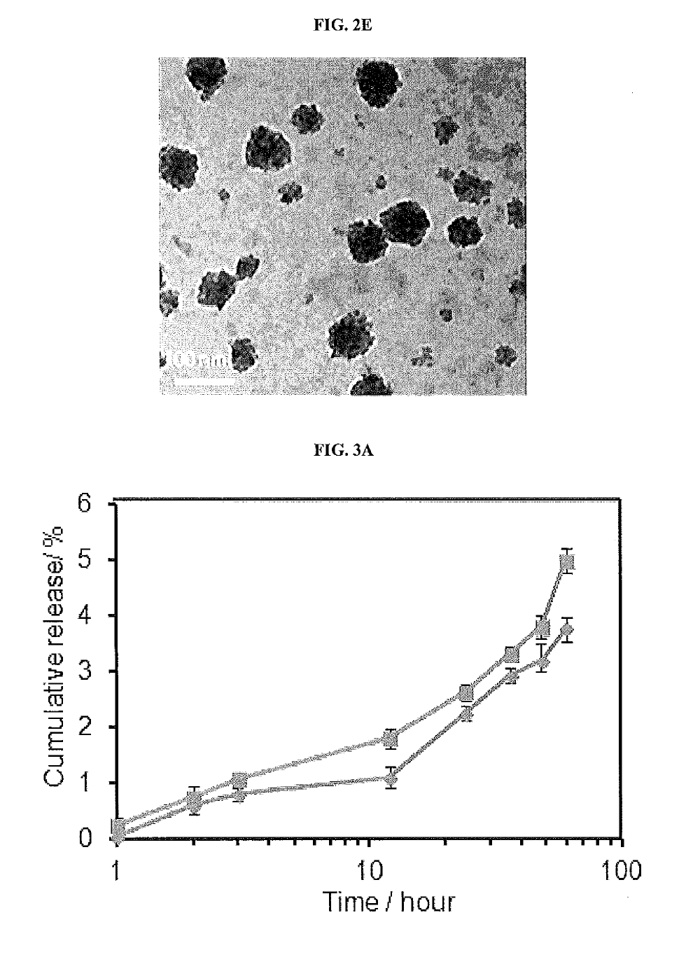

Topology of the polymers may be simply tuned by varying molar ratio of triamine to bisacrylamide. Equimolar ratio of disulfide-based bisacrylamide and triamine in Michael-addition polymerization led to linear reduction-responsive poly(amido-amine) containing secondary and tertiary amines. The disulfide bonds in poly (AMPD-BAC) backbone were sensitive to reducing agents. It may thus be expected that they would be easily cleaved into small molecules in the presence of stimuli. The gel permeation chromatography (GPC) measurements confirmed the cleavage of disulfide bonds of poly(AMPD-BAC) after incubation with GSH to yield lower molecular species as shown in FIG. 2A. The peak intensity of small molecules around 22 min of elution time increased together with the decrease of peak intensity of polymer fraction with high molecular weight (around 17 min), indicating that the polymer degraded into smaller molecules.

Poly(AMPD-CAB) having numerous secondary amine group in backbone offers multiple sites for the attachment of various property modifiers. DTPA was attached to poly(AMPD-CAB) by amidation reaction of secondary amine for complexation of cisplatin. The attachments of PEG was achieved by reaction of secondary amines of poly(AMPD-CAB) with 4-nitrophenyl carbonate activated PEG.

PEG stealth layers may significantly reduce non-specific adsorption of proteins, enabling longer in vivo circulation duration, which in turn improve delivery efficacy. The secondary amines readily react with 4-nitrophenyl carbonate activated PEG to form PEG-grafted poly(AMPD-BAC). The AMPD-BAC unit number per PEG was five as determined by ¹H-NMR measurement.

Diethylene triamine pentaacetic acid (DTPA) was incorporated through formation of amide bond between carboxylic acid group and the remaining secondary amine of poly (AMPD-BAC)-g-PEG backbone. It was used as a chelate for cisplatin to form a carboxylate platinum complex in which two chloride ligands of cisplatin are displaced by carboxylate ligands. The efficiency of incorporation of cisplatin into DTPA unit was found to be approximately 16% (w/w) as determined by ICP-MS. After complexation of platinum complex into the poly(AMPD-BAC)-g-PEG-DTPA, a yellow powder was obtained which provides a first evidence of cisplatin to complex to poly(AMPD-BAC)-g-PEG-DTPA. The complexation was also confirmed by the zeta-potential measurement. The zeta potential of poly(AMPD-BAC)-g-PEG-DTPA was −33.7 mV and poly(AMPD-BAC)-g-PEG-DTPA-Pt was −2.45 mV. The reduction in negative charge indicated that the carboxylic acid group was complexed with cisplatin.

As determined by dynamic light scattering (DLS), poly(AMPD-BAC)-g-PEG-DTPA-Pt forms a nanoparticle with an average diameter of 117.68 nm in aqueous solution with an increase in size of poly(AMPD-BAC)-g-PEG (average diameter: 97 nm) (FIG. 2B). Poly(AMPD-BAC)-g-PEG-DTPA-Pt has an ideal particles size for blood vascular imaging as the optimum nanoparticle size to gain maximum blood circulation time is around 80-150 nm.

The morphology of nanoparticle was visualized by transmission electron microscopy (TEM). Unlike poly(AMPD-BAC)-g-PEG, non-spherical micelles was first obtained for poly(AMPD-BAC)-g-PEG-DTPA (FIG. 2C to FIG. 2E) due to the increase in hydrophilicity of polymer by DTPA. As indicated by the TEM images in FIG. 2E, spherical nanoparticle was able to reversibly form after complexation of platinum complex due to the binding of cisplatin to carboxylic acid group of DTPA, and thus the hydrophilicity of polymer was adjusted to form spherical nanoparticle with increased size and no aggregation was observed upon complexation of platinum complex. The smaller sized nanoparticle of poly(AMPD-BAC)-g-PEG-DTPA-Pt observed by TEM as compared to that measured by DLS can be attributed to the evaporation shrinkage of the PEG shell.

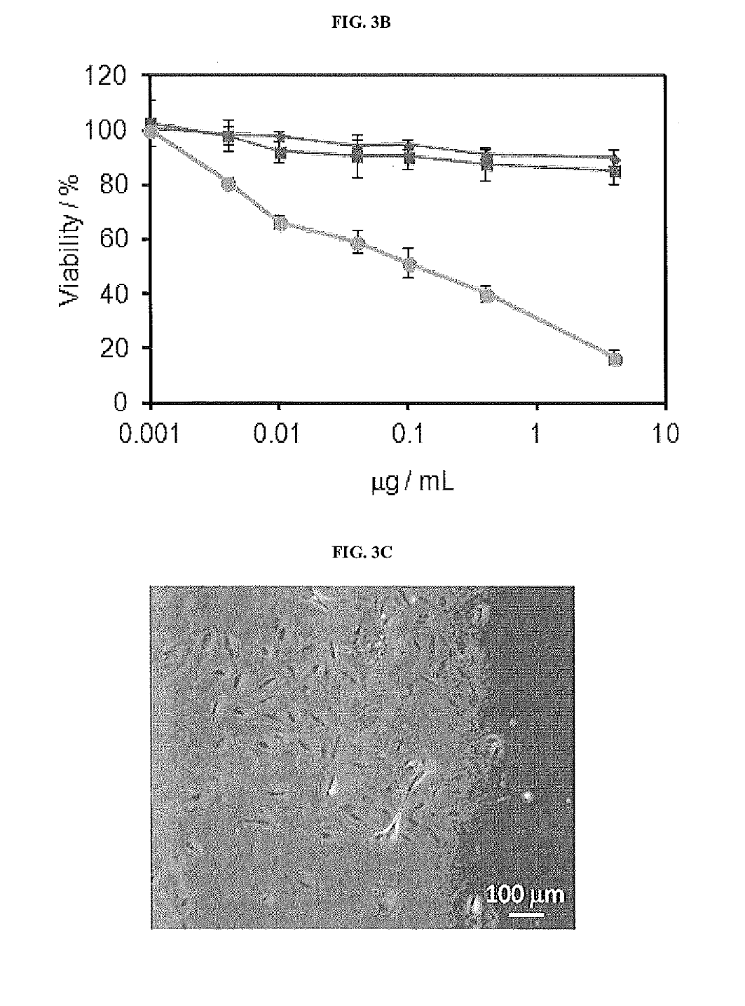

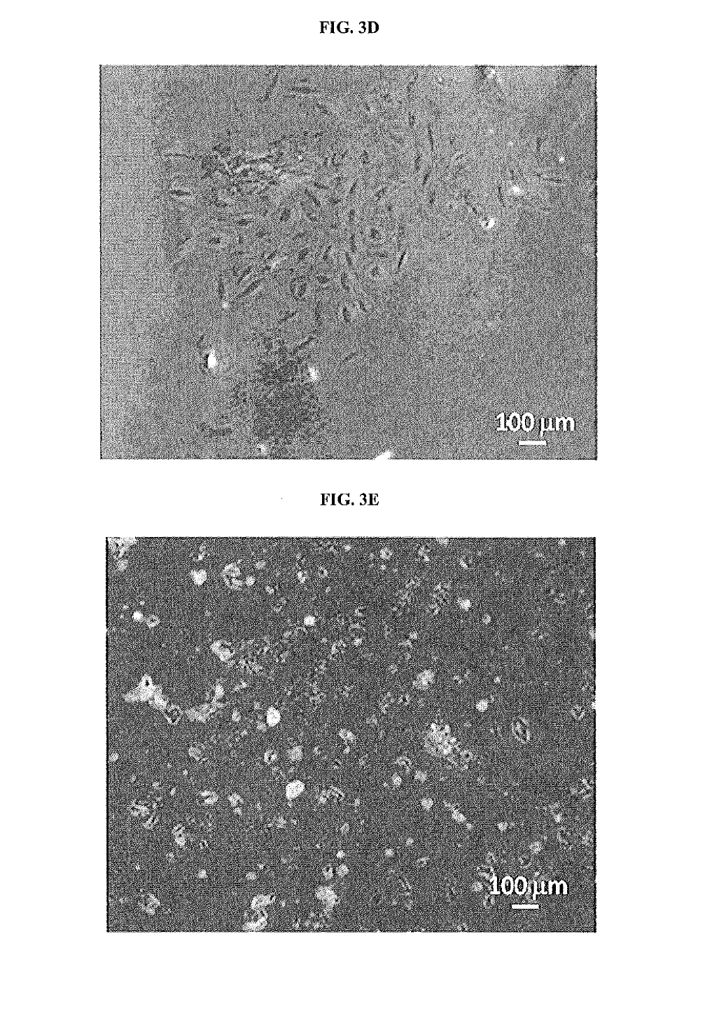

Stability of carboxylate platinum complex in nanoparticle was subsequently evaluated. The obtained release profile of cisplatin exhibited a slow release of platinum complex, at a similar rate in water, with less than 5% of platinum complex content released after 60 h in chloride ion media, and poly(AMPD-BAC)-g-PEG-DTPA-Pt remained yellow in colour, indicating that majority of the platinum complex remained bound with polymer (FIG. 3A). These results are mostly attributable to the chelate in which DTPA is an excellent chelating agent and thus is expected to form a relatively stable carboxylate platinum complex. For this nanoparticle to be used as a contrast agent, its cytotoxicity was evaluated. Both toxicity results of poly(AMPD-BAC)-g-PEG-DTPA-Pt and poly(AMPD-BAC)-g-PEG nanoparticle against on normal breast cell line (MCF-10A) were similar, suggesting that the cytotoxicity of poly(AMPD-BAC)-g-PEG-DTPA-Pt is not significant (FIG. 3B). These results were also reflected in the morphology of cells (FIG. 3C to FIG. 3E). The great stability of platinum complex in nanoparticle clearly rules out any cytotoxic effect from platinum complex.

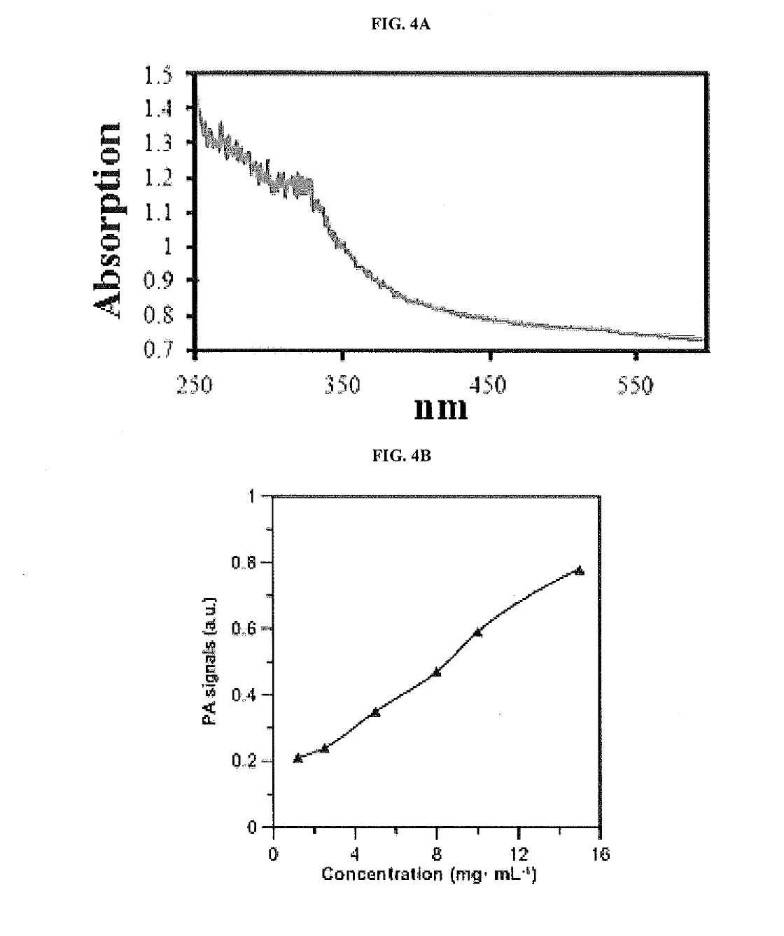

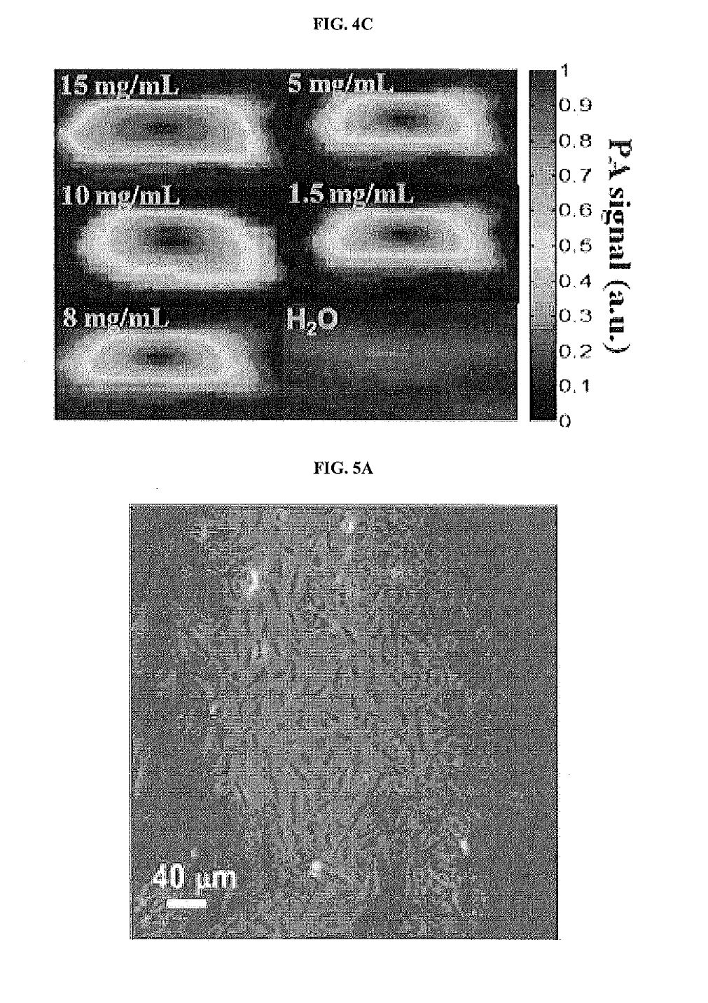

The PA measurement was performed in visible region. The generated photoacoustic signal is due to electronic transitions in the central platinum metal which was assigned to d-d transitions. The results show that poly(AMPD-BAC)-g-PEG-DTPA-Pt can provide high PA signal in which PA of poly(AMPD-BAC)-g-PEG-DTPA-Pt is 3.7 times higher than water (FIG. 4). The investigation of concentration dependence of poly(AMPD-BAC)-g-PEG-DTPA-Pt, PA signals have been measured with a series of poly(AMPD-BAC)-g-PEG-DTPA-Pt samples ranging from 1.2 to 15 mg/mL, and the results are shown in FIG. 4. A linear response is shown for different concentrations samples.

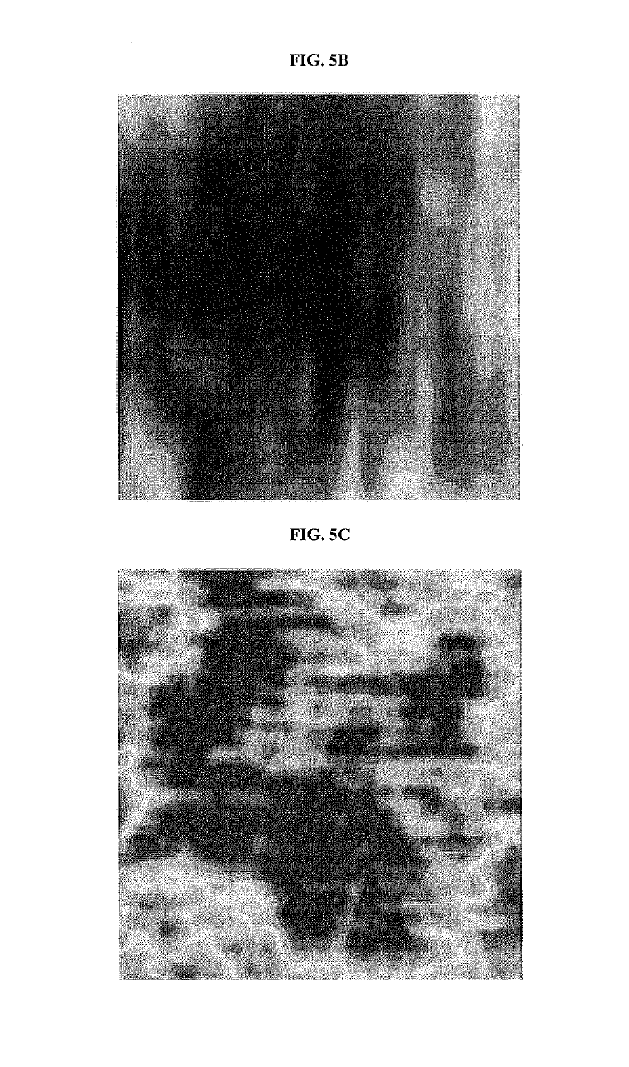

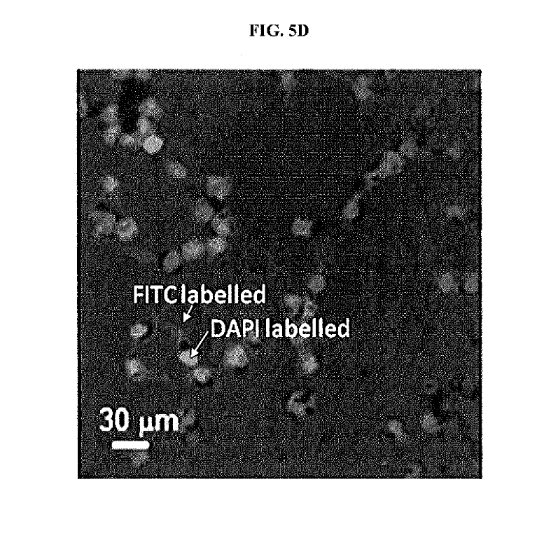

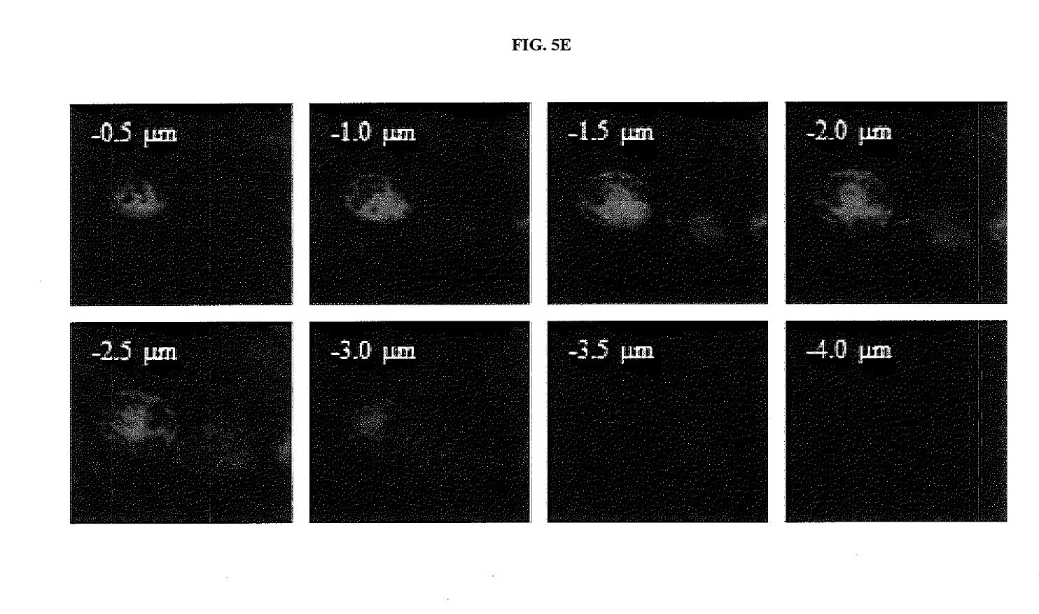

To further demonstrate the value of poly(AMPD-BAC)-g-PEG-DTPA-Pt as a PA contrast agent, MCF-10A cells were incubated with the contrast agent and excess contrast agents were removed prior to PA imaging. As shown in FIG. 5, there are no intrinsic PA signals pre-incubation, but after incubation with poly(AMPD-BAC)-g-PEG-DTPA-Pt (FIG. 5B), clear PA C-scan images of MCF-10A can be observed as shown in FIG. 5C.

Interaction of poly(AMPD-BAC)-g-PEG-DTPA-Pt with cells was further confirmed through visualization via fluorescence confocal microscopy using FITC-labelled poly(AMPD-BAC)-g-PEG-DTPA-Pt. The results in FIG. 5D show that MCF-10A cells showed strong fluorescence, suggesting that poly(AMPD-BAC)-g-PEG-DTPA-Pt may be easily transported into cells. It also provides a clear indication that poly(AMPD-BAC)-g-PEG-DTPA-Pt is distributed in cytoplasm and nuclear region. In order to validate the possibility of internalization of poly(AMPD-BAC)-g-PEG-DTPA-Pt into nucleus over envelopment, depth profiling experiment was conducted. FIG. 5E shows that the poly(AMPD-BAC)-g-PEG-DTPA-Pt has penetrated into the nucleus with uneven distribution. All images were taken from a stack of slices scanned through the cells with a full scanning depth of approximately 5 yin and a scanning step of 0.5 μm. The observation suggests that poly(AMPD-BAC)-g-PEG-DTPA-Pt entered the cell and nucleus.

The application of poly(AMPD-BAC)-g-PEG-DTPA-Pt was further demonstrated in animal. The open-skull window of the rat cortical surface was shown in FIG. 6B. The superior sagittal sinus (SSS) and other vessels on the cortical surface could be observed visually. The SSS is the largest vein in the rodent brain cortex. Imaging of cerebral venous especially SSS is important for traumatic brain injury study.

Although SSS may be imaged by PA microscopy, the detected PA signals from the SSS were weaker than those from other cortical vessels. The inventors have envisaged that photoacaoustic contrast agents may significantly enhance the inherently poor image contrast of SSS. The potential of enhancing SSS detection is to provide a means to study SSS-related cerebral issues. As shown in FIG. 6, the rat cerebral cortex was imaged by custom-designed PAM before and after a single injection of poly(AMPD-BAC)-g-PEG-DTPA-Pt (5 mg/mL). Compared to the brain image based on the intrinsic optical contrast (FIG. 6C), the image acquired 20 min after the administration of poly(AMPD-BAC)-g-PEG-DTPA-Pt shows the brain vasculature with greater clarity, especially the SSS (FIG. 6C), suggesting the strong ability of poly(AMPD-BAC)-g-PEG-DTPA-Pt to generate PA signal in vivo for PA imaging of brain vessels of live rat as well as a low brain parenchymal background. It is known that polymeric agents pose significantly prolonged and enhanced blood pool imaging. The absorption signals from the PAM images acquired at each time point after injection of poly(AMPD-BAC)-g-PEG-DTPA-Pt were integrated and normalized to the signal integration of the pre-injection image. As shown in FIG. 6D, the highest PA signal was acquired at 20 min after a single injection of the contrast agent (5 mg/mL) and the intensity was about 34% higher than that of the pre-injection value. The absorption enhancement remained high, even after 60 min, with an average 23% increase over the pre-injection value which indicates a sufficient amount of poly(AMPD-BAC)-g-PEG-DTPA-Pt circulating in the blood.

The degradation process of disulfide group is relatively fast and can happen within minutes to hours. This is significantly faster than the degradation kinetics of other functionalities such as esters and carbonates, in which degradation of both functional groups require days to weeks. The degradability of poly(AMPD-BAC)-g-PEG-DTPA-Pt was evaluated in which poly(AMPD-BAC)-g-PEG-DTPA-Pt was incubated in the thiol solutions mimicking the plasma composition of the endogenous thiols. Samples were collected at different time points and ultrafiltrated with a filter with a molecular weight cutoff of 2000 Da. The percentage of unfiltrated Pt was determined by ICP-MS and calculated to represent the relative degradability of poly(AMPD-BAC)-g-PEG-DTPA-Pt. As shown in FIG. 6E, poly(AMPD-BAC)-g-PEG-DTPA-Pt degraded rapidly during 8 hours incubation, whereas little degradation was determined in thiol-free solution. Taken together, the poly(AMPD-BAC)-g-PEG-DTPA-Pt with multiple disulfide bonds resulted in strong contrast enhancement in the blood vessel. The disulfide bonds facilitated the excretion of platinum chelate.

The inventors have demonstrated, for the first time, a biodegradable PA contrast agent using a combination of platinum complex and biodegradable polymer. The property of biodegradation and strong PA signal offers a versatile approach for brain vascular imaging which may easily be extended to other transition metal complex and biodegradable polymer due to availability of multiple sites for attachment of various ligands as property modifiers. Advantages of using biodegradable polymer include its intriguing properties such as longevity, intracellular penetration, and versatility of incorporating transition metal complexes. Given that, it can be a useful platform to develop PA contrast agent catered to the specific requirements of preclinical and clinical applications.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A photoacoustic imaging contrast agent comprising a biodegradable polymer backbone grafted with a photoacoustic contrast agent.

2. The photoacoustic imaging contrast agent according to claim 1, wherein the biodegradable polymer backbone is further grafted with at least one of a targeting moiety or a moiety that modulates at least one of bioavailability or half-life of the biodegradable polymer.

3. The photoacoustic imaging contrast agent according to claim 1, wherein the biodegradable polymer backbone comprises a disulfide bond.

4. The photoacoustic imaging contrast agent according to claim 1, wherein the biodegradable polymer backbone is formed by polymerization of a monomer comprising two or more amine groups and a monomer comprising two or more vinyl groups.

5. The photoacoustic imaging contrast agent according to claim 4, wherein the monomer comprising two or more amine groups is an aminoalkyl piperidine.

6. The photoacoustic imaging contrast agent according to claim 4, wherein the monomer comprising two or more vinyl groups is a bisacryl compound.

7. The photoacoustic imaging contrast agent according to claim 6, wherein the bisacryl compound is a bisacrylamide.

8. The photoacoustic imaging contrast agent according to claim 7, wherein the bisacrylamide is N,N′-bis(acryloyl)cystamine.

9. The photoacoustic imaging contrast agent according to claim 8, wherein the monomer comprising two or more amine groups is 4-aminomethyl piperidine.

10. The photoacoustic imaging contrast agent according to claim 1, wherein the biodegradable polymer backbone has repeating units of the general formula 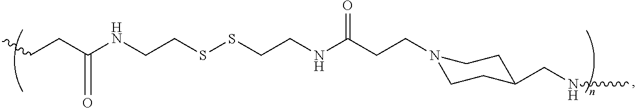
wherein n is an integer in the range of about 1 to 1000.

11. The photoacoustic imaging contrast agent according to claim 2, wherein the photoacoustic contrast agent, the targeting moiety, and/or the moiety that modulates at least one of bioavailability or half-life of the biodegradable polymer are each coupled to a secondary amine group in the polymer backbone.

12. The photoacoustic imaging contrast agent according to claim 1, wherein the photoacoustic imaging contrast agent has repeating units of the general formula 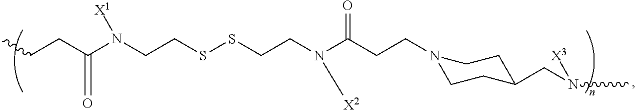
wherein
X¹, X², and X³ at each occurrence is independently selected from the group consisting of H, a photoacoustic contrast agent, a targeting moiety, and a moiety that modulates at least one of bioavailability or half-life of the biodegradable polymer, wherein at least one occurrence of at least one of X¹, X², or X³ is a photoacoustic contrast agent, and
n is an integer in the range of about 1 to about 1000.

13. The photoacoustic imaging contrast agent according to claim 12, wherein the photoacoustic contrast agent is a transition metal complex.

14. The photoacoustic imaging contrast agent according to claim 13, wherein the transition metal complex has the general formula -L¹-M, wherein L¹ is a chelating ligand and M is a transition metal.

15. The photoacoustic imaging agent according to claim 14, wherein the chelating ligand is selected from the group consisting of an aminopolycarboxylic acid, a succinimidyl ester, an isothiocyanate, derivatives thereof, and combinations thereof.

16. The photoacoustic imaging contrast agent according to claim 12, wherein the moiety that modulates at least one of bioavailability or half-life of the biodegradable polymer is a polyalkylene glycol.

17. The photoacoustic imaging contrast agent according to claim 12, wherein the targeting moiety has the general formula -L²-P, wherein L² is a bond or a linker, and P is selected from the group consisting of a polyalkylene glycol, folic acid, an antibody, a targeting protein, and a targeting peptide.

18. The photoacoustic imaging contrast agent according to claim 1, wherein the photoacoustic imaging contrast agent has the general formula 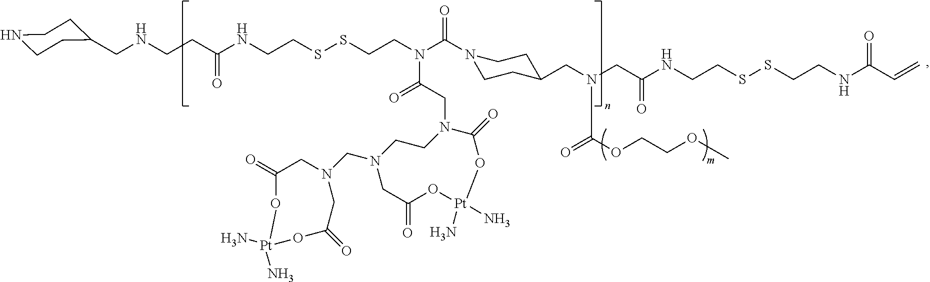
wherein in is an integer in the range of about 1 to 10 and n is an integer in the range of about 1 to about 1000.

19. A method of preparing a photoacoustic imaging contrast agent comprising a biodegradable polymer backbone grafted with a photoacoustic contrast agent, the method comprising
a) providing a biodegradable polymer; and
b) grafting a photoacoustic contrast agent to the biodegradable polymer.

20. A method of imaging living tissue, the method comprising
a) introducing a photoacoustic imaging contrast agent comprising a biodegradable polymer backbone grafted with a photoacoustic contrast agent into living tissue; and
b) obtaining an image of the living tissue by photoacoustic imaging.
